(19) United States
(12) Patent Application Publication
(10) Pub. No.: US 2026/0144573 A1
(43) Pub. Date: May 28, 2026

(54) SURGICAL GUIDE FOR SURGICAL ASSISTED PALATAL EXPANSION

(71) Applicant: Align Technology, Inc., San Jose, CA (US)

(72) Inventors: Jun SATO, San Jose, CA (US); Jeremy RILEY, Mountain View, CA (US); Fan FEI, Mountain View, CA (US); Mitra DERAKHSHAN, Herndon, VA (US)

(21) Appl. No.: 19/401,313

(22) Filed: Nov. 25, 2025

Related U.S. Application Data
(60) Provisional application No. 63/724,893, filed on Nov. 25, 2024.
(60) Provisional application No. 63/862,631, filed on Aug. 12, 2025.

(51) Int. Cl. A61B 17/66; A61C 7/06; A61C 7/10

(52) U.S. Cl. CPC A61B 17/663; A61C 7/06; A61C 7/10

(57) ABSTRACT

Surgical guides (e.g., adjustable splints) that may be used for a temporal expansion and contraction of the palate during and/or after surgical operation on a palatal suture may include first and second tooth-engaging regions and an expandable palatal region including a linkage to allow controlled expansion/contraction of the palatal region. These apparatuses may be configured to allow measurement or estimation of the mobility of the palate (e.g., maxillary segments) and/or locking of the palate in a desired target width.

### CLAIM OF PRIORITY

This patent application claims priority to U.S. provisional Ser. No. 63/724,893, titled “SURGICAL GUIDE FOR SURGICAL ASSISTED PALATAL EXPANSION,” filed on Nov. 25, 2024, and to U.S. provisional Ser. No. 63/862,631, titled “SURGICAL GUIDE FOR SURGICAL ASSISTED PALATAL EXPANSION,” and filed on Aug. 12, 2025; each of these applications is herein incorporated by reference in its entirety.

### BACKGROUND

Splinting the mid-palatal suture following a rapid palatal expansion (RPE) procedure may be an important step to ensure the long-term stability and success of the treatment. For example, rapid palatal expansion may be performed to correct maxillary transverse deficiencies by widening the upper jaw. This process separates the mid-palatal suture, creating space for new bone formation as the suture heals. Without proper stabilization, the suture may relapse or fail to maintain its expanded state, undermining the correction achieved during the procedure.

Adult palatal expansion is a critical intervention for addressing transverse maxillary deficiencies that persist beyond adolescence, when the midpalatal suture has typically fused. In such cases, non-surgical methods are often ineffective, necessitating Surgically Assisted Rapid Palatal Expansion (SARPE). This procedure involves a maxillofacial surgeon performing osteotomies to split the midpalatal suture, enabling skeletal expansion of the maxilla. Devices like the Hyrax™ expander, which use a central screw mechanism, are then employed post-surgery. Initially, the screw is turned to widen the intermolar distance by approximately 3-4 mm, ensuring the two maxillary halves are adequately separated. This step confirms the surgical separation and prepares the site for gradual expansion. After this initial activation, the screw is reversed to return the segments to their original positions, allowing for a controlled, incremental expansion, typically one turn per day. This slow progression supports the biological process of bone remodeling and regeneration, as new bone fills the gap between the separated palatal halves, stabilizing the expansion and improving long-term outcomes.

Hyrax™ expanders, while commonly used in Surgically Assisted Rapid Palatal Expansion, presents several notable disadvantages when applied in adult cases. As a tooth-borne appliance, it transmits expansion forces through the teeth rather than directly to the skeletal structures, which can lead to dental tipping, root resorption, and gingival recession. These effects are particularly concerning in adults, whose periodontal support may already be compromised. Additionally, the Hyrax™ can cause buccal cortical bone loss and increased tooth mobility, especially in patients with periodontal disease or insufficient dental anchorage. The expansion it produces is often non-parallel, meaning the two halves of the maxilla may not separate evenly, potentially leading to asymmetry. Furthermore, in cases where the maxillary sinus extends inferiorly, the effectiveness of the Hyrax is reduced, and complications may increase. These limitations have led to the development of bone-borne or hybrid expanders, which apply force directly to the palatal bone, offering more controlled and stable skeletal expansion with fewer dental side effects.

Described herein are methods and apparatuses that may assist in splinting a mid-palatal suture following a rapid palatal expansion.

### SUMMARY OF THE DISCLOSURE

The methods and apparatuses described herein may be used for adult palatal expansion procedures, in which a surgical procedure incisions are made in the upper jaw, separating the two halves of the palate. This procedure is performed on older patients to assist in quickly expanding the palate. Currently there is no way to guide or confirm the incisions made are sufficient to fully expand the palate. This confirmation would be particularly helpful to simplify the procedure and improve patient outcomes. The apparatuses described herein may permit the surgeon to check that the incisions are adequate to expand the palate, while supporting (or splinting the jaw). In some examples the apparatus may apply a controlled force (in a predetermined direction, e.g., vector) to move the palate and may also return the then return palate to a desired configuration (e.g., unexpanded) to allow incremental expansion. Alternatively, these apparatuses may be used to hold (e.g., splint) the palate in the desired fully expanded configuration. These apparatuses may be held over the palate by being worn on the teeth, without requiring fixation (e.g., bonding) to the teeth or to the palate.

The methods and apparatuses (e.g., devices, systems, etc.) described herein may include one or more surgical guides. These surgical guides may be used with any oral surgery, including but not limited to palatal expansion (e.g., rapid palatal expansion), surgically assisted palatal expansion, modifications of the mid-palatal suture, corticotomy, etc. The surgical guides described herein may include two or more pieces (e.g., a left piece and a right piece); in some cases three or more, e.g., a three-piece maxillae, may be used.

In general, these apparatuses may include a pair of tooth receiving regions configured to be worn over the teeth on the left side of the upper jaw and the right side of the upper jaw. For example, these apparatuses may include a first tooth receiving portion and a second tooth receiving portion. In some cases the tooth receiving portions may cover the occlusal surface of the tooth as well as the lingula side and the buccal side of the teeth. In some cases the tooth receiving portions (e.g., first and second tooth receiving portions, which may correspond to left and right tooth receiving portions) may be coupled to an adjustable, and in some cases locking, palatal region. The palatal region may be formed of two or more portions that may move (e.g., laterally) relative to each other to adjust the width of the palatal region between the first and second tooth engaging regions.

In some examples the adjustable palatal region may include a bias configured to drive expansion of the palatal region laterally between the first and second tooth engagement regions. This bias may be configured to apply a controlled force which may be nonzero, but may be limited to less than, e.g., 15N (e.g., 14N or less, 13N or less, 12N or less, 11N or less, 10N or less, 9N or less, 8N or less, 7N or less, 6N or less, 5N or less, 4N o less, 3N or less, 2N or less, 1N or less, etc.). This force may be applied when the apparatus is in an unlocked configuration allowing expansion or contraction of the palatal region, from an initial position, which may be similar or identical to the patient's initial palatal width, to a target position, e.g., an expanded configuration, in which the patient's palate is expanded by a desired amount (e.g., between about 1.5 mm and 8 mm, between about 2 mm and 7 mm, between about 2 mm and 6 mm, between about 2 mm and 5 mm, etc.).

These apparatuses may be fabricated in any appropriate manner, including by three-dimensional printing (e.g., 3D printing). For example, the surgical guides described herein may be used for a temporal expansion and contraction of the palate after surgical operation on a palatal suture to be split into right and left halves for upcoming rapid palatal expansion and stabilization.

For example, described herein are apparatus for splitting a palatal expansion, the apparatus comprising: a first tooth receiving portion; a second tooth receiving portion, wherein the first and second tooth receiving portions are configured to move laterally closer and/or further apart relative to each other; and a coupling region between the first tooth receiving portion and the second tooth receiving portion, wherein the coupling region is configured to lockably adjust the lateral position between the first and second tooth receiving portions. The coupling region may comprise a rotatable bearing surface. In some cases the coupling region comprises a linear bearing surface. The coupling surface may comprise a plurality of locking positions corresponding to a defined change in a separation distance between the first and second tooth receiving portions. Any of these apparatuses may include one or more slots on the first and/or second tooth receiving portions configured to allow lateral movement but to constrain moment outside of the lateral axis. The first and second tooth receiving portions may be formed by 3D printing.

Also described herein are methods of making and using these apparatuses. For example, described herein are methods including methods of splinting a palatal expansion, the method comprising: inserting an apparatus for splitting a palatal expansion by placing a first plurality of teeth from a right side of an upper jaw of a patient into a first tooth-receiving portion of the apparatus and placing a second plurality of teeth from a left side of the patient's upper jaw into a second tooth-receiving portion of the apparatus; and adjusting the lateral spacing between the first and second tooth receiving portions by adjusting a coupling region between the first and second tooth receiving portions and securing the relative lateral positions of the first and second tooth receiving portions by engaging a lock of the coupling region that prevents lateral movement of the first and second tooth receiving portions relative to each other. In some cases the apparatus is inserted after performing a surgical palatal expansion procedure on the patient. In some cases the method may include adjusting the lateral spacing between the first and second tooth receiving portions a second time. For example, adjusting the lateral spacing may comprise rotating a rotatable bearing surface between the first and second tooth receiving portions, and/or adjusting a linear bearing.

For example, described herein are apparatus for assisting in an oral surgery, the apparatus comprising: a first tooth-receiving portion configured to engage with teeth on a first side of a patient's upper jaw; a second tooth-receiving portion configured to engage with teeth on a second side of the patient's upper jaw, wherein the apparatus is removably wearable over the patient's upper teeth without bonding; an expandable and retractable palatal region extending between the first and second tooth-receiving portions, the palatal region comprising: a first palatal segment coupled to, or integrally formed with, the first tooth-receiving portion; a second palatal segment coupled to, or integrally formed with, the second tooth-receiving portion; one or more linkages movably connecting the first and second palatal segments; and a lock configured to selectively fix the relative position of the first and second palatal segments.

The linkage may include one or more rails, channels, hinges, arms, or rack-and-pinion mechanisms. In some examples the linkage comprises a rotatable bearing surface or a linear bearing surface. The linkage may comprise a plurality of locking positions corresponding to defined lateral separations between the first and second tooth-receiving portions. The linkage may include a biasing element, is configured to apply a predetermined lateral expansion force between the first and second palatal segments. For example, the biasing element may apply a force of less than 10 Newtons.

In general, any of these apparatuses may include one or more indicators configured to display the degree of lateral expansion of the palatal region. The first and second tooth-receiving portions may include occlusal cut-out regions to allow intercuspation with the lower jaw. The palatal region may be configured to provide curvilinear expansion between the first and second palatal segments. The apparatus may be fabricated using additive manufacturing.

The lock may include a pin-and-socket, latch, or sliding lock. The apparatus or method may include a control configured to adjust the lateral spacing between the first and second palatal segments, the control comprising one or more of: a dial, a knob, a lever, or a switch. He control may be configured to switch between a guide mode and a splinting mode. The apparatus may be formed of a polymeric material. The linkage may comprise one or more elongate members having a trapezoidal cross-section.

Also described herein are methods of making and/or using any of these apparatuses as described herein. For example, a method of assisting in palatal expansion in a patient may include: performing one or more incisions of the patient's palate and/or adjacent regions to allow repositioning of maxillary segments forming the palate; monitoring expansion of an apparatus, wherein the apparatus comprises a first palatal segment coupled to a first tooth-receiving portion worn on one or more teeth in the patient's upper jaw and a second palatal segment coupled to a second tooth-receiving portion worn on a second one or more teeth in the patient's upper jaw, wherein the first and second palatal segments are movably coupled via one or more linkages between the first and second palatal segment; and stopping or adjusting the incisions based on the spacing between the first and second palatal segments.

Any of these methods may include applying a biasing force between the first and second palatal segments to expand the patient's upper palate and/or confirming mobility of the maxillary segments based on the lateral expansion achieved. Any of these methods may include locking the spacing between the first and second palatal segments to stabilize the maxillary segments. Any of these methods may include inserting the apparatus onto the patient's teeth. The apparatus may be inserted after performing an initial incision. Stopping the incisions based on the spacing between the first and second palatal segments may comprise stopping the incision after confirming mobility of the maxillary segments. Any of these methods may include returning the spacing between the first and second palatal segments to an initial or near-initial configuration after confirming mobility of the maxillary segments. Any of these methods may include incrementally adjusting the spacing between the first and second palatal segments over time to gradually expand the palate. Adjusting the spacing between the first and second palatal segments comprises rotating a dial, knob, or lever on the apparatus.

Monitoring expansion of the apparatus may comprise observing an indicator on the apparatus to determine the amount of lateral expansion. Any of these methods may include assessing intercuspation of the patient's upper jaw with the patient's lower jaw through occlusal cut-out regions in the apparatus. The apparatus may be worn continuously during and after the surgical procedure. Any of these methods may include switching the apparatus from a guide mode to a splinting mode and/or selecting a target expansion spacing between the first and second palatal segments and adjusting the apparatus to achieve the selected spacing.

Any of these methods may include method be configured to include an apparatus that is removably worn over the teeth without bonding to the teeth or palate.

For example, described herein are methods of treatment planning for an oral surgical procedure. In some examples that the method comprising: accessing or receiving a three-dimensional digital model of the patient's maxillary dentition and palatal anatomy having an initial width; accessing or receiving a target palatal expansion for the patient's maxillary dentition and palatal anatomy; generating a surgical guide apparatus comprising: a first tooth-receiving portion configured to engage with teeth on a first side of the patient's upper jaw; a second tooth-receiving portion configured to engage with teeth on a second side of the patient's upper jaw; an expandable and retractable palatal region extending between the first and second tooth-receiving portions, wherein the apparatus has a first configuration wherein the expandable and retractable palatal region is configured to expand the patient's palate to the target palatal expansion and a second configuration wherein the expandable and retractable palatal region is configured to restore the patient's palate to the initial width; wherein the surgical guide apparatus is configured to be switched from the first configuration to the second configuration. Accessing or receiving the target palatal expansion may comprise simulating palatal expansion using the digital model to determine the target palatal expansion. In general, generating the surgical guide apparatus comprises fabricating the surgical guide apparatus using additive manufacturing. Any of these methods may include the surgical guide apparatus comprises generating a digital model of the surgical guide apparatus. In some cases these methods may include generating a surgical treatment plan including using the surgical guide apparatus to guide and confirm palatal expansion. The surgical treatment plan may include wearing the surgical guide apparatus for a period of time following the surgery to splint the patient's palate. Any of these methods may include incorporating a biasing element into the apparatus to apply a predetermined expansion force during simulation or surgery.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1 schematically illustrates an example of an apparatus for assisting in an oral surgery as described herein, such as an apparatus for splinting a palatal expansion (e.g., an adjustable splint).

FIG. 2 schematically illustrates an example of an apparatus for assisting in an oral surgery as described herein.

FIG. 3 schematically illustrates an example of an apparatus as described herein.

FIG. 4A schematically illustrates an example of an apparatus for assisting in an oral surgical procedure, such as palatal expansion, as described herein.

FIG. 4B shows examples of a cross-sectional geometries of linkages that may be used with the apparatuses described herein.

FIGS. 5A-5C schematically illustrate an example of an apparatus for assisting in an oral surgical procedure as described herein. FIG. 5A shows a perspective view, FIG. 5B shows a top view and FIG. 5C shows a front view.

FIG. 6 schematically illustrates an example of an apparatus as described herein including two handles.

FIGS. 7A-7C schematically illustrate an example of an apparatus as described herein including cut-out occlusal regions. FIG. 7A shows a perspective view, FIG. 7B shows a top view and FIG. 7C shows a front view.

FIGS. 8A-8D illustrate types of relative movement that may be achieved by an apparatuses as described herein. FIG. 8A shows an initial position. FIG. 8B shows the segments of FIG. 8A in a laterally expanded configuration. FIG. 8C shows the segments of FIG. 8A laterally expanded by different amounts (resulting in a triangular expansion). FIG. 8D shows the segments of FIG. 8A in a laterally curving expansion.

### DETAILED DESCRIPTION

In general, these apparatuses can be configured to test expansion of the maxillary segments during a surgical procedure, such as, but not limited to a SARPE procedure in which one or more incisions are made to allow repositioning of maxillary segments forming the palate. These methods and apparatuses may be used as a measurement tool during the medical procedure, testing the amount of movement possible by the palate (e.g., maxillary segments) and/or guiding the movement. These methods and apparatuses may allow the user to select the amount of separation (using a control, such as a dial, level, etc.) on the apparatus. These method and apparatuses may output the amount of separation of the maxillary segments, for example, using a visual output that is calibrated on the apparatus to indicate the amount of expansion of the apparatus (and therefore the maxillary segments). The methods and apparatuses described herein may therefore be used during the surgery and/or after the surgery. For example, the apparatus may be configured to be worn on the teeth during and/or after a surgery to incise the palate or adjacent regions to allow movement of the maxillary segments. Alternatively or additionally, any of these apparatuses may be configured to stabilize the palate following the surgery. For example, the same or a different device may be worn over the teeth to hold the teeth in sequentially expanded (from an initial position) positions. Thus, any of these apparatuses may be configured to splint the palate to hold it securely to allow healing and bone growth during the expansion.

When used during a surgical procedure to allow movement of the maxillary segments, the apparatus may be configured to stabilize during the procedure, e.g., when the doctor is cutting. Thus, it can be a guide during and/or after the procedure. Thus, in some variations the apparatus may be used during the surgery. The apparatus may provide additional feedback about the relative shape and/or size of the expansion of the palate and may guide the surgeon in performing the procedure. Any of these apparatuses may include cut-out regions over the occlusal surface(s). In some configurations, this may permit examination of the intercuspation between the upper and lower jaws during the procedure to allow adjustments during surgery. When the apparatus is configured for use post-surgery, the occlusal cut-out regions may enhance the patient's bite and may be more comfortable. As mentioned, any of these apparatuses may be configured to lock the relative positions of the palatal regions during and/or after the surgery. The same apparatus may be used for different expansion steps, e.g., in variations having incremental expansion. In particular, the apparatuses described herein may be configured for use during surgery (e.g., to assist in the surgical procedure) and immediately following surgery, which may prevent having to remove and adjust the apparatus, which may be difficult and may increase the chances of harm to the patient.

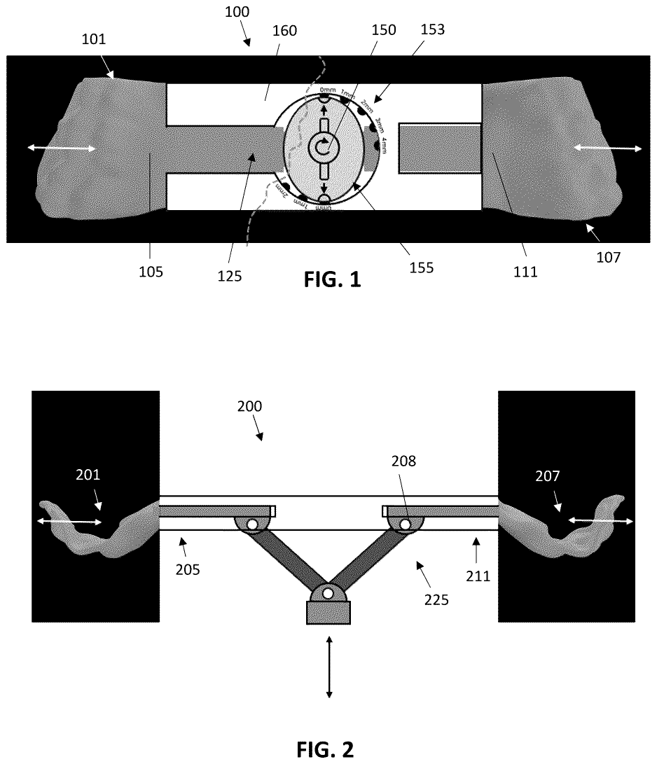

Thus any of these apparatuses may include a lock, allowing the patient to wear the apparatus immediately after the procedure. For example, the apparatus may be configured to lock the palatal regions back in the original un-expanded configuration, similar to the initial configuration or just incrementally expanded. The variation worn home could be separate from the version used during surgery. The surgical variations may have occlusal cut-outs to allow confirmation of jaw alignment. The version worn home by the patient may not include occlusal cut-outs. The apparatus may include feedback showing an indicator of the separation. See, e.g., FIG. 1. In some cases, the clinician (doctor, surgeon, etc.) may select a predetermined separation (e.g., 1 mm, 2 mm, 3 mm, 4 mm, 5 mm, 6 mm, 7 mm, 8 mm, 9 mm, 10 mm, etc.).

The product could have one or more controls, including controls to set or adjust the separation between the palatal first and second palatal regions. Any appropriate control may be used, including a dial, knob, pin, lever, switch, etc. The control may configurability adjust the separation between the palatal regions. In some cases the control may include a neutral setting to allow the distance between the palatal regions to float. In some cases the control may be configured to lock the separation between the palatal regions at a predetermined value. In some cases the control may be configured to limit the separation to a maximum, such as, e.g., 2 mm, 2.5 mm, 3 mm, 3.5 mm, 4 mm, 4.5 mm, 5 mm, 5.5 mm, 6 mm, 6.5 mm, etc. In some examples the surgeon or other dental professional may set or prescribe the amount of separation and configure the apparatus to provide this separation amount. Any control may be used, including (but not limited to) a dial, bias (e.g., spring, etc.), slider, etc. In some cases the apparatus may include a control to switch between modes including a guide mode (e.g., for use during a surgical procedure) in which the control allows the apparatus to adjust/change the spacing between the palatal regions; this mode may include a bias, e.g., applying force tending to separate the palatal region, e.g., to a maximum separation. In some cases the control may be configured to select a locked mode in which the spacing between the palatal regions is locked. This spacing may be preset or selected from a number of spacing options (e.g., 1 mm, 1.5 mm, 2 mm, 2.5 mm, 3 mm, 3.5 mm, 4 mm, 4.5 mm, 5 mm, 5.5 mm, etc.). In some apparatuses the control may be configured to adjust between the surgical mode (e.g., the guide mode) and the locked mode (e.g., locked at a single spacing between the palatal regions). A splint, e.g., in the form of a fixed orthodontic appliance or a removable retainer, serves to immobilize the expanded suture while the surrounding bone remodels and consolidates. This stabilization allows the body sufficient time to deposit new bone tissue in the created gap, ensuring the structural integrity and permanence of the expansion. Additionally, splinting prevents the recurrence of the original malocclusion or the collapse of dental arches, which could compromise both functional and aesthetic outcomes.

For example, in general any of the apparatuses described herein for assisting in a surgical procedure, including but not limited to palatal expansion, may be configured to be removably worn over the subject's (e.g., the patient's) upper teeth and palatal region. As used herein “removable” may include patient-removable and/or physician removable. In some cases the apparatuses are configured so that the patient may remove them, e.g., for cleaning, changing, etc. In some cases the apparatus may be configured to be secured to the patient so that a dental professional may remove it, but not (in most cases) the patient.

Any of the apparatuses described herein may include a first tooth receiving portion and second tooth receiving portion. As mentioned above, the first and second tooth received portions may be configured to fit over teeth on opposite sides of the patient's upper jaw (maxillary teeth) and may each include a cavity configured to fit over the teeth on that side of the jaw (e.g., the premolars and molars, and in some examples, the canines. In some examples the apparatus may also be configured to include a third tooth receiving portion configured to hold the incisors. In general the apparatuses described herein are configured to be customized to a particular subject's teeth. For example, the apparatuses described herein may be generated by scanning a patient's dentition, e.g., using an intraoral scanner, generating and/or modifying a three-dimensional model of the patient's teeth, and fabricating the appliance using any appropriate manner, including by automatic fabrication (e.g., 3D printing, etc.).

In general, the first and second tooth receiving portions may include at least a buccal side and a lingual side that, in some examples, may extend from the occlusal side over the full occlusal and/or lingual side and over at least a portion of the gingiva when worn.

These apparatuses may also include a laterally expandable/retractable palatal region extending between, and coupled to, the first and second tooth receiving portions. The palatal region may include two or more regions, e.g., a first palatal region and a second palatal region. The first and second palatal region may be movably and/or lockably coupled together. For example, any of these apparatuses may include one or more linkages movably connecting the first palatal region and the second palatal region, The one or more linkages may include one or more of: a rail, a channel, a hinge, an arm, a rack and pinion, etc. In some cases the one or more linkages may be configured one or more rails extending between and coupling the first and second palatal regions. The first palatal region may be coupled to and/or may be integrally formed with the first tooth receiving portion and the second palatal region may be coupled to and/or may be integrally formed with the second tooth receiving portion. In some cases the linkage may be configured to stabilize the movement between the first and second palatal regions forming the laterally expandable/retractable palatal region. For example, the one or more linkages may comprise a rail having a trapezoidal cross-section forming a dovetail geometry. An of these apparatuses may include a linkage having one or more hinges.

The linkages may be configured to guide the expansion and contraction of the palatal region in use. For example, the linkage may be configured to provide lateral movement between the first palatal region and the second palatal region. In some cases the posterior region between the first and second palatal regions may expand laterally more than the anterior region between the first and second palatal regions. In some cases the one or more linkages may be configured to allow lateral and out-of-plane movement between the first and second palatal regions.

Any of these apparatuses my include a lock configured to secure the first and second palatal regions to prevent relative movement between the first and second palatal regions. Any appropriate locking mechanism may be included. For example, the lock may be a pin and socket, a latch, etc.

In general, these apparatuses may be configured to allow intercuspation with the teeth of the lower jaw, which may further enhance the ability of the surgeon when incising the tissue to adjust the palatal region (e.g.,. maxillary segments). For example, the first and/or second tooth receiving portions of any of these apparatuses may include occlusal openings that are configured to expose the occlusal surface of the teeth within the tooth receiving regions to allow intercuspation with the occlusal surfaces of teeth on the opposite jaw. The rim around the occlusal opening may be thickened to provide support. In some cases, this rim may be reinforced.

Any of the apparatuses described herein may include one or more controls. For example, the apparatuses described herein may be configured to have separate modes of operation. For example, these apparatuses may include a first mode in which the apparatus is configured to expand the palatal region as surgery is performed and/or immediately after the surgery to indicate how mobile the maxillary segments are (e.g., for a SARPE procedure, to see if they are sufficiently free to move for palatal expansion), and/or to limit the movement to a maximum amount. In this mode the apparatus may include an indicator configured to indicate how much expansion (e.g., in mm, degrees, etc.) has occurred. The apparatuses may also include a second mode in which the palatal region is locked in a particular expansion amount, and/or may be controllably adjusted to a particular expansion amount. In some cases this expansion amount may be less than the maximum amount. In some cases, the expansion amount may be the initial palatal width or a slightly increase palatal width (e.g., 0.5 mm expansion, 1 mm expansion, 1.5 mm expansion, etc.). the apparatuses described herein may include a control to switch between the first and second modes. The same or a different control may be configured to lock the palatal region (e.g., to engage the lock) to limit palatal expansion to a predetermined separation. For example, the control may be configured to switch between a first state that permits relative movement between the first palatal region and the second palatal region and a second state that prevents relative movement between the first palatal region and the second palatal region; in some cases the second state corresponds to an initial separation between the first and second palatal regions.

These apparatuses, particularly those having the first state in which the device maximally expanded, may include one or more biases that is/are configured to expand the first and second palatal regions with a predetermined force. This predetermined force may be, e.g., 10 N or less, 9 N or less, 8 N or less, 7 N or less, 6 N or less, 5 N or less, 4 N or less, 3 N or less, 2 N or less, 1 N or less, 0.5 N or less, etc.). Any appropriate bias may be used, including a spring, elastomeric material, etc. The bias may be coupled to the linkage to apply the force to laterally expand the palatal region (e.g., in the first mode).

As mentioned, any of these apparatuses may be formed by an additive manufacturing technique (e.g. 3D printing). In some cases the apparatus may be at least partially formed of a polymeric material.

The second mode may be referred to herein as splinting mode. In general, these apparatuses may act as a splint to hold or secure the apparatus in a fixed position. Proper splinting is particularly important in palatal expansion. In adults, where the suture is more rigid, splinting may ensure the success of palatal expansion, which may be referred to specifically as rapid palatal expansion (RPE), by counteracting the natural tendency for soft tissue and muscle forces to revert the expansion. By maintaining the suture in its expanded position during the healing phase, splinting supports the long-term effectiveness of the procedure, contributing to improved oral health, better bite alignment, and enhanced airway functionality.

As is often the case with rapid palatal expansion for adult cases, the surgical operation to split the mid palatal suture is needed for skeletal expansion because the suture is already fused; not only splitting the suture but also expansion of the arches, which sometimes includes cuts on the buccal cortical plate. In the case of contemporary commercially available expansion devices (e.g., with a turnkey), doctors or caregivers typically turn the screw to increase the intermolar width (˜3-4mm) to make sure the two halves are disconnected/loosen after the surgical operation and, subsequently, they unturn the screw to bring them back to the original positions to start gradual expansion (˜1 turn/day) to assist the biological response (bone remodeling/filling the gap between two halves).

With removable palatal expanders (e.g., IPEs) for surgical assisted cases, there is currently no means to temporarily expand the palate to 3-4 mm, which would be useful to initially confirm that the maxillary segments are sufficiently mobile following incisions, because the IPE consists of a series of separate removable devices for incremental expansion. If one tried to wear later stages (e.g. stage #11 of 12 or more stages), the device does not fit to the dentition because of the significant amount of dimensional mismatch.

Described herein are apparatuses that may act as surgical guides (e.g., adjustable splints) that may be used for a temporal expansion and contraction of the palate during and/or after surgical operation on a palatal suture to be split into right and left halves for upcoming rapid palatal expansion. Examples of schematics illustrating these apparatuses are shown in FIGS. 1-6, below. These apparatuses may be made in any appropriate manner, including but not limited to 3D printing (e.g. an SLS process), and no assembly processes for separate components are required. Those devices may consist of multiple moving parts.

Any of these apparatuses may include two or more (e.g., three or more) rigid components, such as a first (e.g., left) tooth-receiving region configured to engage with the patient's teeth, a second (e.g., right) tooth-receiving region configured to engage with the patient's teeth and a movable coupling between the two configured to move the first and second regions laterally towards or away from each other (e.g., in a wider/narrower direction of the upper jaw). In some cases the movable coupling is configured as a bearing surface (e.g., rotary bearing surface) that may be rotated to alter the separation between the first and second regions.

For example, FIG. 1 shows one example of an apparatus 100 (e.g., a device) as described herein including separate left and right halves including a first tooth receiving portion 101 and a second tooth receiving portion 107. The first tooth receiving portion is integrally formed with a first palatal region 105 and the second tooth receiving portion is integrally formed with the second palatal region 111. These first and second palatal regions are engaged with each other through a linkage 155 that is formed from a rotating oval rotor at the center, and a housing 160. The housing provide a channel in which a plunger-like portion of the linkage extends through. The ends of each plunger-like arm that contacts the oval rotor 155. A control 150 is configured as a knob that can be rotated to turn the oval rotor to adjust the width of the palatal region by moving the first and second palatal regions relative to each other. In some cases the apparatus includes an anterior portion (e.g., 3 or more pieces). In this example the rotor rotates inside a circular stator in the housing. The housing, stator and arms form the linkage. When the rotor rotates, the oval rotor pushes the plunger to increase or decrease the intermolar width to split the suture. The plunger arms may be biased open and/or closed. Any appropriate rotor may be used. In some cases the rotor can be a screw that is embedded. The stator may have one or more notches where each notch represents the expansion amount (e.g., 1 mm, 2 mm, 3 mm, etc.), and the rotor can stop at each increment and the doctor can haptically feel the stop to precisely control the expansion amount. Once the desired expansion is achieved the doctor rotates the dial to zero position and the plungers come back to the original positions due to the maxillary relapse contraction force.

The apparatus shown in FIG. 1 may also include a lock to lock the linkage in a predetermined position or positions (including those marked on the indicator 153 shown on the control/rotor. In general, these apparatuses may include one or mor indicators (markings, color coding, text, numbers, etc.) indicating the width (e.g., degree of expansion) of the palatal region. The indicators may be on the palatal region (e.g., the lingual side of the palatal region).

In some cases the movable members between the first and second tooth-receiving portions may be configured as an arm or arms forming part of the linkage. For example, FIG. 2 shows an example of a variation of an apparatus 200 including a linkage 225 having multiple hinges 208, which connected an arm to each half of the device, e.g., on a first palatal region and a second palatal region (e.g., the first and second tooth-receiving portions), which may slide inside the housing like a linear bearing. The first palatal region 205 and the second palatal 211 region are coupled to or integrally formed with a first tooth receiving portion 201 and a second tooth receiving portion 207, respectively. The two halves separate as the top vertical control (e.g., knob) gets driven up. The sliding housing can have notches to allow the dental professional to determine the travel distance in more friendly manner (as shown in FIG. 1). Once the desired expansion is achieved, one can retract the halves by lifting up the nob back.

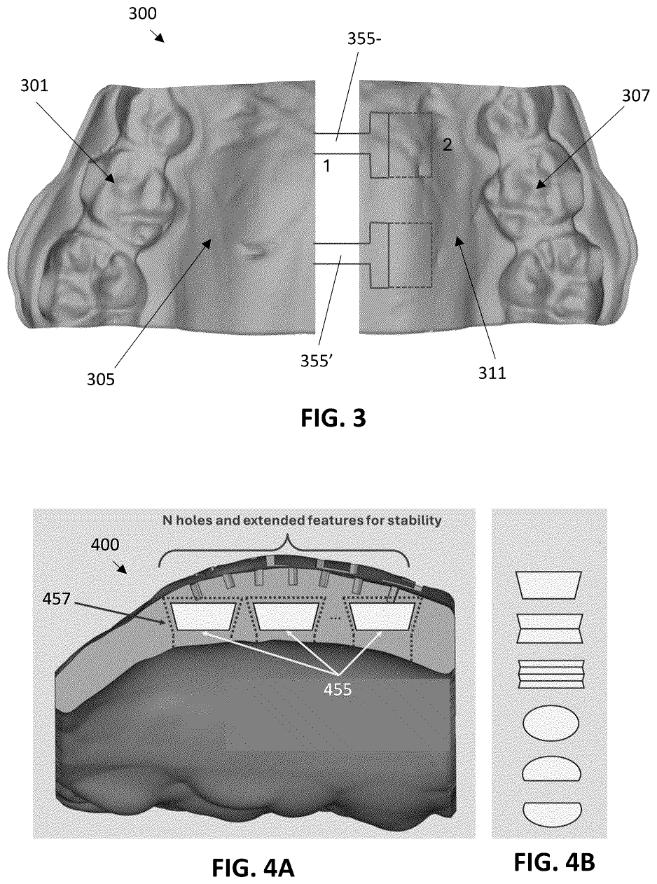

FIG. 3 shows an example of an apparatus 300 which includes a first tooth receiving portion 301 integrally formed with a first palatal region 305, a second tooth receiving portion 307 integrally formed with a second palatal region 311, so that the overall palatal region is split into two halves. A pair of linkages, configured as elongate members 355, 355′ extend from the first palatal region into receiving channels in the second palatal region 311. Thus, the linkage may slide to expand/contract the lateral length of the palatal region. In some cases the apparatus may include one or more piece in the anterior papilla area or pre-maxillary to control movement in this direction as well. The receiving channel may be open (or partially open, having an open diameter less than the diameter of the elongate member so that it does not come out of the channel) on one side or may be fully enclosed. In some cases the linkage may include a stop which keeps the palatal region from expanding too much (e.g., fully separating the first and second palatal regions). This apparatus may be readily 3D printed.

FIG. 4A illustrates an example of a section through an apparatus 400 that is similar to the variation shown in FIG. 3, in which a plurality of linkages (three are shown) 455 connect the first palatal region from the second palatal region. The plurality of linkages may provide enhanced stability of movement in transverse direction. In some cases the cross-section of the elongate members forming the linkage 455 are trapezoidal, forming dovetail-like geometries for smooth sliding movement within the linkage channel, which may also help prevent the elongate members from falling out of the sliding channels (e.g., slots). The slots may have similar dovetail geometries and can be bottom-open 457 or top-open, so that the residual material in them can be cleaned after fabrication. FIG. 4B shows examples of different cross-sectional geometries that may be used to help retain the elongate member of the linkage in the channel, including trapezoidal, waisted, multiply waisted, oval, clipped oval, etc.

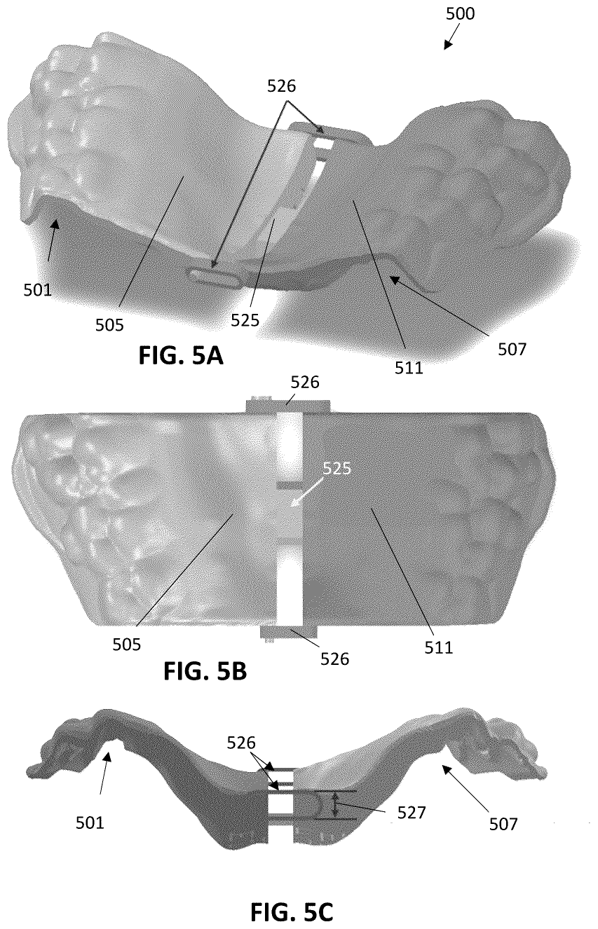

FIGS. 5A-5C show an example of an apparatus as described herein having stabilizing external slots 526. In this example, the first palatal region 505 (connected to the first tooth-receiving portion 501) and the second palatal region 511 (connected to the second tooth-receiving portion 507) are configured to slide relative to each other over one or more linkages 525 and may be locked in a predetermined or selectable lateral position. For example, in FIGS. 5A-5C, the stabilizing external slots 526 may act as locking guide features and may include stopping points that can be positioned on the apparatus. The linkage may include an elongate member (e.g., rail, track, etc.) that includes dovetail features, such as a trapezoidal cross-section that enhance stability of the apparatus. As shown in FIG. 5C, the height of the external guides 527 (external slots 526) may be smaller than height of the apparatus.

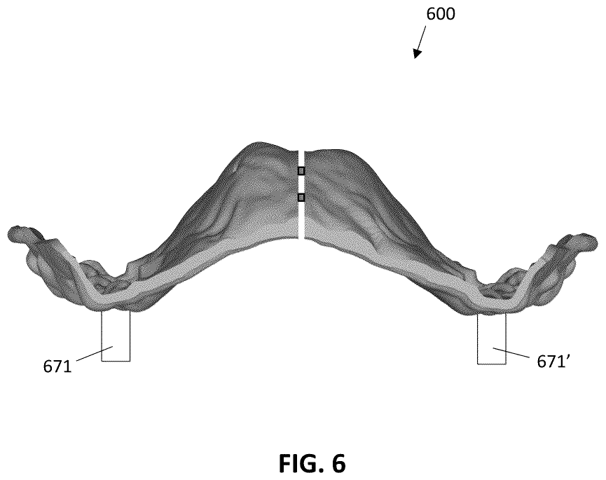

FIG. 6 shows an example of an apparatus 600 including handles 671, 671′ that may be included with any of the apparatuses described herein, including those shown in FIGS. 1-5C. The handles may give the dental professional something to hold when doing the expansion/contraction. For example, handles or grips may extend from one or both of the first/second palatal region and/or the outer surface of the first and/or second tooth receiving portion, and may be used to manually expand or contract (or assist in expansion/contraction) of the apparatus. Thus, these handles may help with performing surgical procedures, including assessing the mobility of the maxillary segments when incising the tissue. In some examples the handles may be used for temporal expansion in non-surgical cases as well. For instance, when performing a class correction, e.g. Class III correction, the doctor or other dental professional can use this handles to expand and retract the arch to loosen the mid-palatal suture, and to begin class III correction more effectively (e.g. with facemasks, elastics, other Class III corrector) for younger patients. For both surgical and non-surgical embodiments, the apparatuses described herein may include differential thickness on the occlusal plan; for example if a surgery involves vertical movements. In some variations the apparatus does not include the occlusal prints of the teeth (e.g., the outer surface of the occlusal region) but may have a flat (or flatter) surface, or may be configured to enhance grip (e.g., including handle or other gripping regions, to help restrict articulation if it is desirable to stabilize the jaw independently. However, if it is desirable to stabilize the upper and lower in occlusion the apparatus may include occlusal groves that match the actual or desired configuration of the occlusal surface of the upper (maxillary) teeth, which may help align with the lower teeth (e.g., the mandibular dentition). In some examples, two or three points or regions on the occlusal surface may be used for stabilization. Alternatively, as described below, the actual occlusive surfaces of the teeth may be used, by including one or more occlusal cut-out regions.

### Checking Fit with Lower Arch

Any of these apparatuses may be configured to include cut-out regions over the occlusal region of the tooth-receiving portion to allow analysis of the jaw intercuspation.

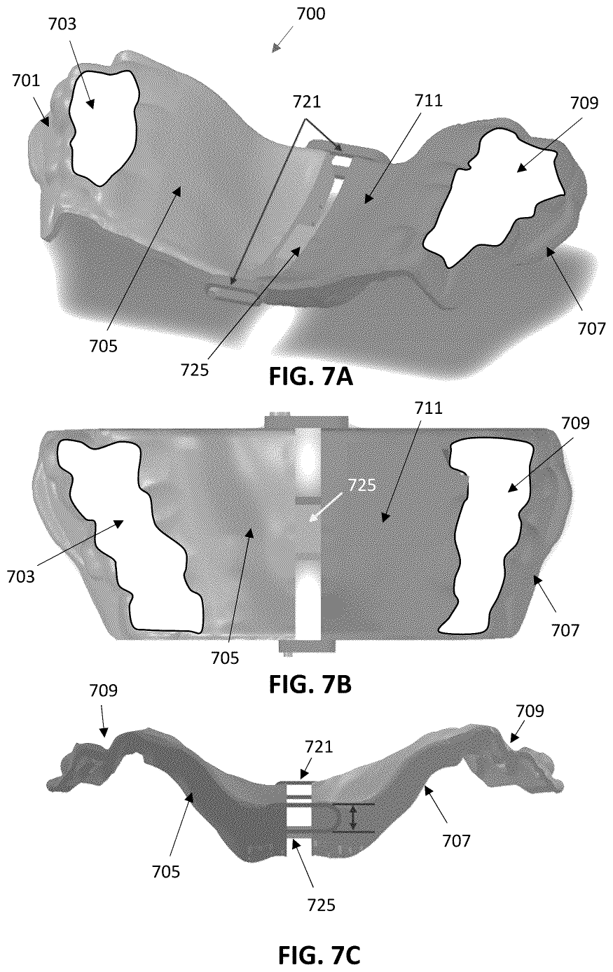

FIGS. 7A-7C illustrate an example of an apparatus 700 similar to that shown in FIGS. 5A-5C in which the occlusal region(s) of both tooth receiving portions 701, 707 have been cut out to allow the occlusal regions 703, 709 to engage with the teeth on the lower jaw (e.g., mandibular teeth). In FIG. 7A the apparatus 700 includes a first tooth engaging region 701 configured to engage with teeth on the left side of the patient's upper jaw; the second tooth engagement region 707 also includes a cut-out region 709 that is configured to expose the occlusal region of the teeth. The first and second palatal regions 705, 711 are configured to move laterally relative to each other by sliding over linkage 825 that extends between the first 705 and second 711 palatal regions. In some cases the apparatus may include additional guides such as slots 721 as described in reference to FIGS. 5A-5C. The guide or guides 725 may be keyed to reinforce and/or strengthen the bearing surface between the first and second palatal regions, as described in FIGS. 3 and 4A-4B.

Any of these apparatuses may include one or more features (not shown), such as a bite ramp, bite block, etc. to engage with the lower jaw or a device worn on the lower law.

In any of these apparatuses, a patient could wear the apparatus during and/or immediately following the surgery to show prior to and after the surgery.

### Locking Mechanism

As mentioned, any of these apparatuses may include a lock (e.g., locking mechanism) that may hold the apparatus in a set position of lateral expansion. For example, FIGS. 5A-5C may include a lock that may secure the relative positions of the lateral members (left side and right side). In some cases the locking mechanism may be used with these sliding configuration to secure the separation between the two. The lock may lock the position in an expanded configuration (e.g., expansion by 3 mm) and/or a smaller separation.

In general, these apparatuses are worn on the teeth but not bonded to the teeth, allowing them to be removed and easily swapped out or replaced if necessary, or for cleaning, etc.

In FIG. 1 the apparatus may include a lock (e.g., locking mechanism) that may be configured as a locking pin or other element that engages with a receiver to prevent relative movement of the palatal regions. The lock may hold either or both of the palatal regions, e.g., to a guide or other region spanning between the palatal regions. In some cases the lock is a sliding lock. The apparatus may include a plurality of locking positions. For example, the apparatus may include a first locking position holding the apparatus in the expanded position (e.g., a fully expanded position). In some cases the apparatus includes a lock in the patient initial position.

FIGS. 5A-5C also illustrate an example of a lock, as described above, included as part of an external guide 526.

### Measurement Tool

Any of these apparatuses may also be configured to measure and indicate the amount of movement and/or separation of the maxillary segments. For example, apparatus may be configured to include an output that indicates the movement of the maxillary segments, e.g., the extent or degree of the separation/expansion of the palate. In some cases these apparatuses may be configured to determine the degree of separation as the incisions are made, by applying a force (e.g., a biasing force) to drive separation from the initial position to a final position.

In some cases, such as in the example device shown in FIG. 1, the arrow may point to the amount of additional separation, in either discrete (set distances) or in a continuous manner. For example, the apparatus may be configured in some cases to include a control (e.g., dial, button, knob, lever, etc.) which may drive and/or limit expansion of the two side of the tooth, e.g., between about 0 mm and about 6 mm (e.g., between about 0 mm and about 5 mm, between about 0 mm and 4 mm, etc.).

As mentioned, any of these apparatuses may include a bias to apply force driving the maxillary segments apart, e.g., by driving them laterally apparat with a bias force. The bias force may be relatively low, and an in some cases may be user adjustable.

### Direction of Movement

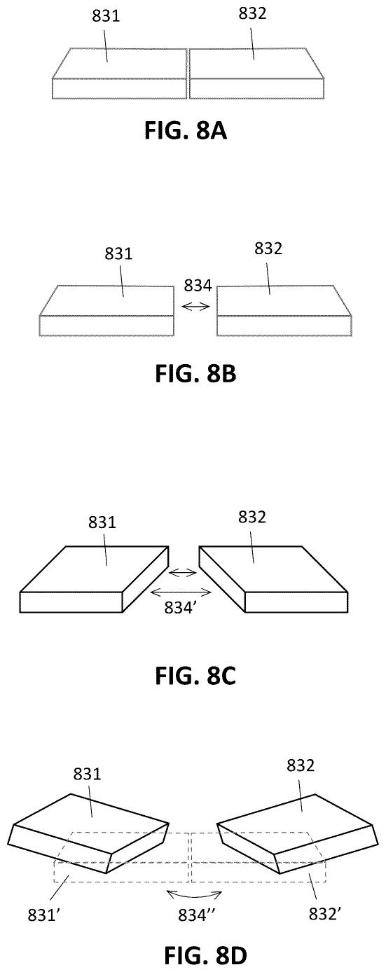

In general, any of the apparatuses described herein may be configured to expand laterally, to widen the palate as described above. Alternatively or additionally, in some cases the apparatus may be configured to move in other directions, which may better track more physiological expansion of the jaws. For example in some cases the apparatus may be configured to pivot or rotate as the two or more portions of the palatal region separate relative to each other. For example, FIG. 8A illustrates a pair of rectangular segments 831, 832 representing the two portions of a palatal region of a device which may be movably connected to each other as described above. For example, the two (or more) palatal regions may be connected to each other by one or more guides (not shown), forming one or more bearing surfaces. In FIG. 8B the two portions are configured so that the two or more portions of the palatal region (each of which is coupled to a tooth receiving portion) move laterally 834 relative to each other, as shown by the arrow. When an apparatus configured to move in this manner is worn by the patient, the lateral movement results in lateral expansion of the palate. In some cases the lateral movement may be non-uniform between the two portions. For example, FIG. 8C shows rectangular boxes representing two portions of a palatal region that are configured to move 834′ so that the more distal region moves less than the more proximal region. In FIG. 8C, the more anterior region moves laterally less than the more proximal region, which may be achieved using two or more curved guides connecting the two regions (e.g., curving in the lateral plane), resulting in an angle between the inner edges of these two regions when expanded. In any of these examples the posterior movement may be larger than anterior movement, and/or the posterior movement may be smaller than anterior movement.

In some cases the two or more regions may move out of the lateral plane in addition to moving in the lateral plane. For example, FIG. 8D shows two portions 831, 832 that are configured to rotate/pivot up (e.g., out of the lateral plane), they separate laterally. This may be achieved by including two or more guides that curve up (e.g. out of the lateral plane) in the direction of movement. In FIG. 8D the dashed shapes 831′, 832′ illustrate the initial position from which the palatal regions are moved into the laterally and out-of-plane curving expansion 834″ shown. In general, these apparatuses may be configured so that the two (or more) portions of the palatal region, relative to each other in any appropriate direction/dimension.

The design of the movement, as well as the extent of this movement, may be set during the planning stage for planning expansion of the patients palate. In any of these apparatuses a bias may be included, as described above, tending to open the palate once the incisions are sufficient to allow movement of the maxillary segments. Any of these apparatuses mya include one or more locking positions and one or more locks to secure the two or more portions of the palatal regions as shown in any of these predetermined positions. Any of these apparatuses may include an indicator showing the relative separation (e.g., in mm, degrees, etc.) between the first and second palatal regions.

In general the palatal region of the apparatuses described herein may be configured to minimally cover the regions of the palate that are being cut by the surgeon, so that these apparatuses may be worn and used during the procedure. In some cases the palatal region may have a smaller anterior-to-posterior width than the tooth receiving portions and/or may include openings or gaps to allow access to the underlying palate.

### METHODS

Described herein are surgical methods that may incorporate any of these apparatuses, including SARPE. For example, in some cases these methods may include performing surgery in which the apparatus is used as a guide during the surgical procedure, e.g., performing the osteotomy, and indicating if, and/or how much, the maxillary segments may shift or move.

For example, in some examples, the medical professional may make incisions in one or more of: the midpalatal suture, and/or in the maxillary vestibule (e.g., above the attached mucosa), and/or between the lateral incisive anteriorly and the second molar region (e.g., posteriorly on both sides), and/or an exposure of the lateral walls of the maxilla, and/or a sulcular incision (e.g., between the central incisive and the first premolar teeth). The procedure may be performed under general anesthesia in an operating room. In general, the apparatus described herein, which may be placed in the mouth before or after the surgical incisions, may be activated to apply a force, which may be predetermined, to move the maxillary segments a predetermined amount, confirming that the surgical incisions are appropriate to widen the gap between the separated bones as desired. In some cases the method may then include returning the maxillary segments to a second position (e.g., the initial position or to a position in which the maxillary segments are moved slightly relative the initial position). Thus, as described herein, the same apparatus may be configured to move the maxillary segments to first position, which may be set and/or predetermined to confirm the surgical incisions are correct and/or complete and may then optionally include actuating the apparatus to return the maxillary segments to a second (e.g., initially or nearly-initial) position. The movement to the first position may be biased so that lateral sides of the apparatus are driven apart with a predetermined force. The second position is typically narrower than the first position. In any of these apparatuses the apparatus may include a control for selecting subsequent additional positions, each subsequent position gradually and sequentially widening the palate, e.g., expanding it. Any of these apparatuses and methods of using them may include locking or holding the apparatus in any of these positions, e.g., by activating a lock. After each adjustment (e.g., each progressively wider position), the patient may be allowed time to heal, so that the palate may fill in the space with new bone growth, resulting in a wider palate.

Alternatively, also described herein are apparatuses and methods in which the surgery is performed and the apparatus may be used to actively adjust the separation between the maxillary segments to confirm that the incisions are sufficient to allow the maxillary segments to move as desired, e.g., by applying a force or range of forces to separate the maxillary segments. In some examples, rather than returning the maxillary segments to the initial (or a near-initial) position, the method and apparatus may instead by configured to hold the maxillary segments at the broadened position, and act as a splint to allow the body to heal and grow into the new position. Thus any of these apparatus may be configured to be worn to hold the maxillary segments in the new position and allow the bone to heal in this new position.

In general, in any of these methods and apparatuses, the apparatus may be configured to allow the surgeon or other medical professional to confirm that the first position, in which the maxillary segments may be expanded as fully as desired, also allows proper engagement with other teeth, including teeth on the opposite jaw (e.g., mandibular teeth) or other oral features (e.g., tongue, lips, etc.). For example, after wearing the apparatus and performing the incisions until the palatal region expands as desired, the occlusal regions of the apparats may be open to allow the medical professional to determine how the upper and lower teeth are positioned relative to each other with the maxillary segments moved to the target position.

In some cases the same apparatus may be configured to have a fixation configuration, which the apparatus is configured to retain the maxillary segments in a position for an extended time period (e.g., 1 day to multiple weeks, be., 10 days or more), and a surgical assisting configuration (e.g., guide, checker, etc.), in which the apparatus is configured to identify if and/or when the bone has been sufficient cut by the procedure to allow movement of the maxillary segments as desired. In some cases separate devices may be used to perform these functions. In some examples the same apparatuses may be used to perform these functions.

These apparatuses may also be used as a splint or stabilizer device for oral surgeries, not limited to palatal expansion. For example, these apparatuses may be used as a guide during any oral surgery and/or after oral surgery for retaining and/or fixation.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

What is claimed is:

1. An apparatus for assisting in an oral surgery, the apparatus comprising:
a first tooth-receiving portion configured to engage with teeth on a first side of a patient's upper jaw;
a second tooth-receiving portion configured to engage with teeth on a second side of the patient's upper jaw, wherein the apparatus is removably wearable over the patient's upper teeth without bonding;
an expandable and retractable palatal region extending between the first and second tooth-receiving portions, the palatal region comprising:
a first palatal segment coupled to, or integrally formed with, the first tooth-receiving portion;
a second palatal segment coupled to, or integrally formed with, the second tooth-receiving portion;
one or more linkages movably connecting the first and second palatal segments; and
a lock configured to selectively fix a relative position of the first and second palatal segments.

2. The apparatus of claim 1, wherein the one or more linkages comprises one or more rails, channels, hinges, arms, or rack-and-pinion mechanisms.

3. The apparatus of claim 1, wherein the one or more linkages comprises a rotatable bearing surface or a linear bearing surface.

4. The apparatus of claim 1, wherein the one or more linkages comprises a plurality of locking positions corresponding to defined lateral separations between the first and second tooth-receiving portions.

5. The apparatus of claim 1, wherein the one or more linkages includes a biasing element, is configured to apply a predetermined lateral expansion force between the first and second palatal segments.

6. The apparatus of claim 5, wherein the biasing element applies a force of less than 10 Newtons.

7. The apparatus of claim 1, wherein the first and second tooth-receiving portions include occlusal cut-out regions to allow intercuspation with the patient's lower jaw.

8. The apparatus of claim 1, wherein the palatal region is configured to provide curvilinear expansion between the first and second palatal segments.

9. The apparatus of claim 1, further comprising a control configured to adjust lateral spacing between the first and second palatal segments, the control comprising one or more of: a dial, a knob, a lever, or a switch.

10. The apparatus of claim 9, wherein the control is configured to switch between a guide mode and a splinting mode.

11. A method of assisting in palatal expansion in a patient, comprising:
performing one or more incisions of the patient's palate and/or adjacent regions to allow repositioning of maxillary segments forming the patient's palate;
monitoring expansion of an apparatus, wherein the apparatus comprises a first palatal segment coupled to a first tooth-receiving portion worn on one or more teeth in the patient's upper jaw and a second palatal segment coupled to a second tooth-receiving portion worn on a second one or more teeth in the patient's upper jaw, wherein the first and second palatal segments are movably coupled via one or more linkages between the first and second palatal segment; and
stopping or adjusting the one or more incisions based on a spacing between the first and second palatal segments.

12. The method of claim 11, further comprising applying a biasing force between the first and second palatal segments to expand the patient's upper palate.

13. The method of claim 11, further comprising confirming mobility of the maxillary segments based on a lateral expansion achieved.

14. The method of claim 11, further comprising locking the spacing between the first and second palatal segments to stabilize the maxillary segments.

15. The method of claim 11, further comprising inserting the apparatus onto the patient's teeth after performing an initial incision of the one or more incisions.

16. The method of claim 11, wherein stopping the one or more incisions comprises stopping the one or more incisions after confirming mobility of the maxillary segments.

17. The method of claim 11, further comprising returning the spacing between the first and second palatal segments to an initial or near-initial configuration after confirming mobility of the maxillary segments.

18. The method of claim 11, wherein monitoring expansion of the apparatus comprises observing an indicator on the apparatus to determine an amount of lateral expansion.

19. The method of claim 11, further comprising assessing intercuspation of the patient's upper jaw with the patient's lower jaw through occlusal cut-out regions in the apparatus.

20. The method of claim 11, wherein the apparatus is worn continuously during and after performing the one or more incisions.

21. The method of claim 11, further comprising switching the apparatus from a guide mode to a splinting mode.

22. The method of claim 11, wherein the apparatus is removably worn over the teeth without bonding to the teeth or palate.
